(19) United States
(12) Patent Application Publication
(10) Pub. No.: US 2003/0172929 A1
(43) Pub. Date: Sep. 18, 2003

(54) Inspired-volume-dependent gas dosage

(76) Inventor: Rainer Muellner, Wiener Neudorf (AT)

Correspondence Address: CONNOLLY BOVE LODGE & HUTZ, LLP, 1220 N MARKET STREET, P O BOX 2207, WILMINGTON, DE, 19899

(21) Appl. No.: 10/149,616

(22) PCT Filed: Dec. 6, 2000
(86) PCT No.: PCT/EP00/12246

(30) Foreign Application Priority Data
Dec. 18, 1999 (DE) 199612064

(51) Int. Cl. A61M 16/00

(52) U.S. Cl. 128/204.18; 128/204.22

(57) ABSTRACT

The gas-supply system for the inhalation treatment of humans or mammals, entailing controlled dosing of at least one gas, characterized by a tidal volume-dependent regulation of the gas dosing.

The invention relates to a gas-supply system for the inhalation treatment of humans or mammals, entailing controlled dosing of at least one gas; it also relates a method for operating the gas-supply system and to its use.

Breathing devices are employed in mechanical ventilation, anesthesia and respiratory therapy calling for treatment with gases such as, for instance, oxygen administration or treatment with nitric oxide (NO).

Patients suffering from chronic breathing difficulties (for example, asthma or chronic obstructive pulmonary disease—COPD) use a normally portable oxygen dispenser to supply oxygen to the body. Such patients are referred to as spontaneously breathing patients, in contrast to patients who are intubated and hooked up to a ventilator in a hospital. Spontaneously breathing patients are given, for example, additional oxygen (LOT=long-term oxygen therapy) or breathing support (via continuous positive airways pressure—CPAP). The gases are administered either via so-called nasal clips or nasal probes (nasal administration; in the simplest case, a gas-supply tube whose opening is positioned open below the nostrils of the patients) or via a breathing mask (especially in the case of CPAP).

WO 98/31282 (internal designation TMG 2028/67) describes a gas-supply system for ventilated or spontaneously breathing patients with which one or more gases (for example, NO, oxygen) are dosed irregularly (continuously or discontinuously) into the breathing gas by a control means (program control, sensor control or combined program-sensor control).

Depending on the level of exertion of the spontaneously breathing patient, her/his tidal volume increase or decreases. As a result of which the respiration rate as well as the characteristics of the breathing curve (inspiration curve) change.

Up until now, it has not been possible to record the inspiration curve of spontaneously breathing patients (open respiratory circulation system) and, at the same time, to dose one or more gases or aerosols. Devices that merely record the depth of the breath at the beginning of the dosing only allow very imprecise conclusions to be drawn about the actual tidal volume since the entire course of the curve is not known. Moreover, particularly under exertion, the entire course of the curve or the curve characteristics can change considerably.

The invention is based on the objective of optimizing the gas dosing in inhalation therapy, especially for spontaneously breathing patients.

This objective is achieved by means of a gas-supply system having the features described in claim 1.

The gas-supply system for the inhalation treatment of humans or mammals comprises a device that serves to dose gases or aerosols, especially medical gases (for example, oxygen, gas containing NO) or aerosols (for instance, asthma drugs). The breathing curve-dependent dosing can be employed for all types of gases (also in a combination), particularly oxygen and a gas containing NO or a gas containing NO and hydrogen; oxygen and hydrogen; oxygen and helium; oxygen, a gas containing NO and hydrogen; oxygen, a gas containing NO and helium; oxygen, carbon dioxide and helium; or oxygen, a gas containing NO, carbon dioxide and hydrogen, as well as aerosols. The gas-supply system with tidal volume-dependent gas dosing, that is to say, the dosing of gases or aerosols, is used for ventilated, or especially preferably, for spontaneously breathing patients.

The basic equipment configuration of gas-supply systems for ventilated or spontaneously breathing patients is described in WO 98/31282 (internal designation TMG 2028/67), to which reference is hereby made.

The gas-supply system for a tidal volume-dependent regulation of the dosing of gases or aerosols preferably comprises an additional gas line fitted with a sensor and leading to the patient (human or mammal). This additional gas line is connected, for example, to a nasal clip or breathing mask. The sensor preferably detects the pressure or gas flow in the nose or mouth area of the patient. The pressure in the nose or mouth area is referred to as respiratory pressure, while the gas flow in the nose or mouth area is designated as breathing gas flow. A breathing curve depicts the course over time of the respiratory pressure or breathing gas flow.

The course of the breathing curve is recorded particularly by measuring the pressure course during one breathing cycle (expiration and inspiration), for example, in or on the nasal clips, normally using a pressure sensor or a flow sensor (or systems based on these). If the breathing curve is measured continuously, especially during the inspiration, the tidal volume at every point in time is known. Moreover, the recording of the breathing curve while the patient is at rest and the noticeable change in the breathing curve allow conclusions to be drawn about the momentary level of exertion of the patient. The change in the tidal volume detected by the sensor is advantageously conveyed to a control unit that then commensurately regulates the amount of gas or aerosol dosed and, for instance, actuates controllable dosing valves so that the dosed amount changes (for example, by leaving the dosing valves open for a longer period of time).

The quantity V of gas or aerosol that has to be dosed or that has been dosed is calculated on the basis of the following formula:

V(mL)=[desired concentration(%)*tidal volume(mL)]/100.

The controlled adaptation of the gas amount to the state of the patient ensures that the gas amount or gas concentration needed for the therapy in question is changed as a function of the change in the tidal volume. For instance, the supplied gas concentration can be kept constant relative to the tidal volume or else the gas quantity or gas concentration can be increased in comparison to the resting rate, based on the ascertained level of exertion of the patient. This means that the dosing device does not keep the concentration of gases in the lung constant, but rather, it increases the concentration in order to increase the effect under exertion.

Another quantifiable criterion for the level of exertion of the patient is the number of breaths per minute.

An evaluation of the parameters tidal volume, number of breaths and characteristics of the inspiration curve allows conclusions to be drawn about the level of exertion of the patient so that the therapy can be adapted accordingly.

The tidal volume is advantageously recorded by means of a second line leading to the patient (nasal clip or mask) in which the momentary pressure is measured during the entire time.

The gas dosing is, for instance, inspiration-synchronized, whereby the duration of the dosing and/or the quantity of gas dosed per unit of time are changed as a function of the ascertained level of exertion of the patient.

The breathing gas flow, particularly the breathing gas flow during inspiration (inspiration flow) is recorded, for example, by measuring the pressure (negative pressure) during the entire inspiration phase, which is proportional to the gas flow or inspiration flow. This negative pressure is advantageously recorded using a relative pressure sensor. Another possibility is to measure the gas flow directly employing a flowmeter.

Any errors that might occur as a result of the gas dosing (positive pressure) are advantageously compensated for by means of algorithms in a control program, as a rule in the control unit. It is particularly advantageous to employ interpolation of the recorded pressure or gas flow curve over time in order to determine the tidal volume.

When several gases (for instance, O₂ and gas containing NO) are dosed, the quantity of one gas can be kept constant while simultaneously, the amount of the second gas is changed. The point in time of the dosing can also be selected at will, since it is precisely defined through the recording of the inspiration curve. Thus, a gas can be dosed at the time of the triggering of the dosing while a second gas is then only dosed later on.

In order to keep the concentration of the gas constant, the amount of gas is varied in such a way that the quantity of supplied gas is adapted to the tidal volume (for example, an increasing amount of gas when the tidal volume rises).

A control valve could also be employed to change the gas flow in the breathing gas line and to adapt it to the individual curve shape.

Thus, a so-called gas spike (momentary gas surge) can be administered so that even when the tidal volume varies, the areas at the site of action (as a rule in the lungs) that are exposed to the flow are always the same.

Another possibility consists of dosing via a control valve so that the dosing flow is adapted to the pressure curve and the gas is dosed in accordance with this pressure curve.

The breath-dependent gas dosing of one or more gases and/or aerosols can generally be employed for all types of dosing control, particularly for program control, sensor control or combined program-sensor control used for inspiration-synchronized gas dosing, which is carried out pulse-modulated or in sequences.

These types of control for gas dosing are described in WO 98/31282 (internal designation TMG 2028/67), to which reference is hereby made.

In order to attain a simultaneous regulation of the gas dosing as a function of the measured tidal volume, the tidal volume is measured, for instance, simultaneously (during the same breathing cycle) and the gas dosing is regulated or the tidal volume of the preceding breathing cycle is employed as the basis for the regulation of the gas dosing for the next breathing cycle.

The invention will be explained in reference to the drawing.

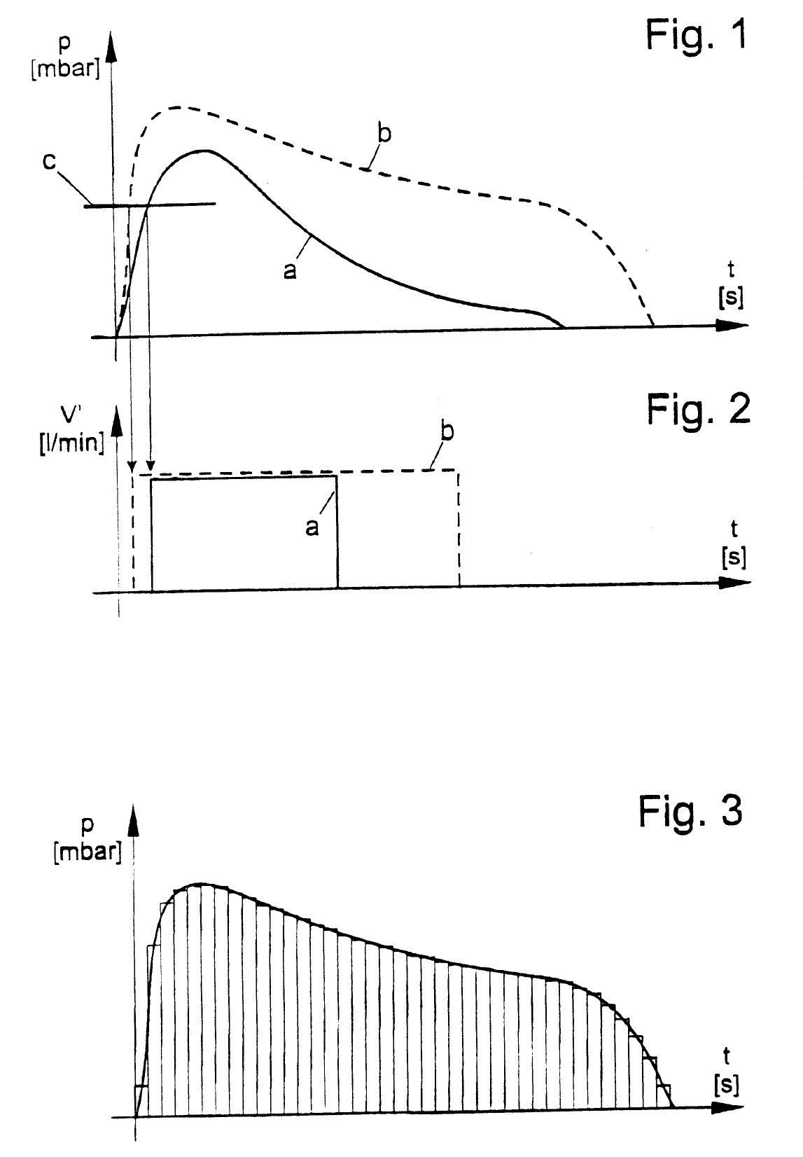

FIG. 1 schematically shows a breathing curve (respiratory pressure P in mbar plotted against the time t in seconds) for the resting state a and for the exertion state b of a patient. The gas dosing is triggered once a specified threshold value (triggering value) c is reached. This is illustrated in FIG. 2. The dosed gas volume flow V′ (in L/min) resulting from the breathing curve-dependent regulation is shown in FIG. 2 for the states a (rest) and b (exertion) as a function of the time t (in seconds).

FIG. 3 schematically shows how the breathing curve is interpolated from individual measured values of the respiratory pressure.

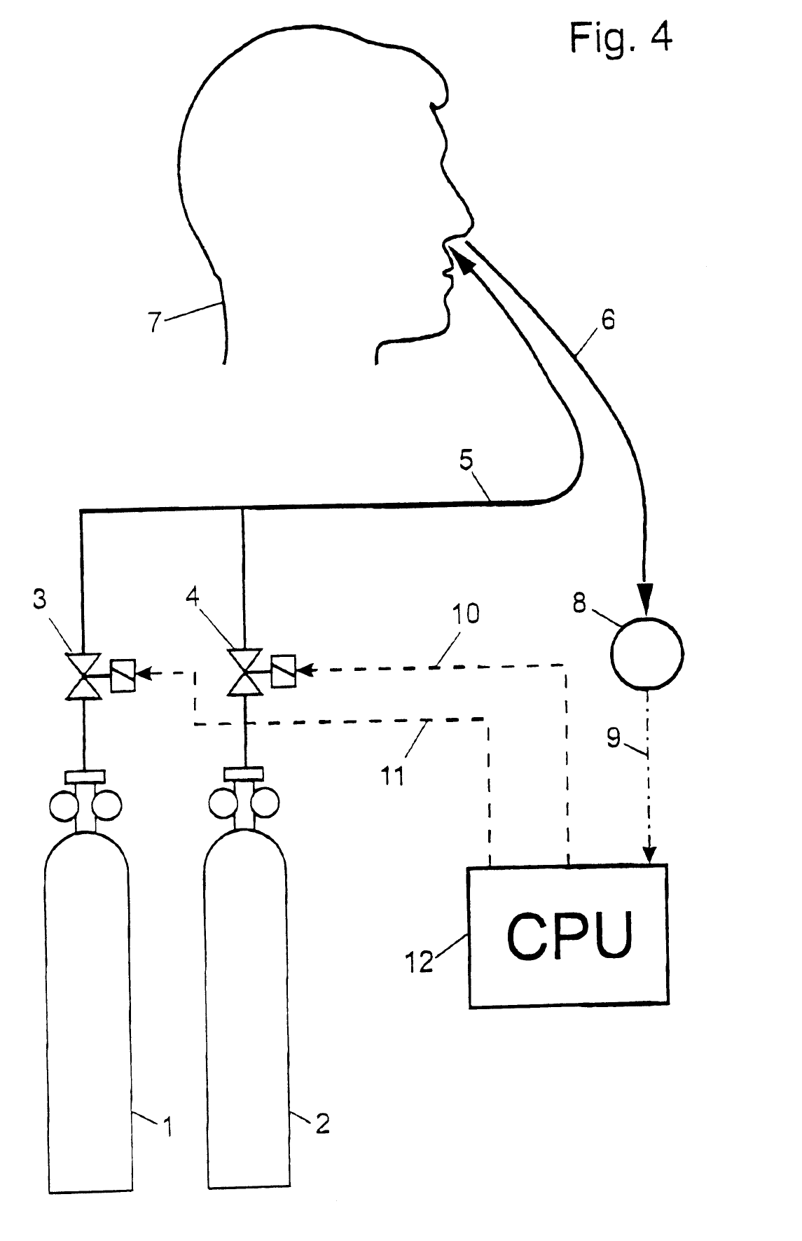

FIG. 4 schematically shows a gas-supply system, especially for spontaneously breathing patients. The gas is dosed via adjustable solenoid valves 3, 4 which are connected to the control unit 12 via control lines 10, 11. The triggering for the dosing is a defined signal of the pressure or flow sensor 8 that is conveyed to the control unit 12 through the control line 9. The gas supply system shown serves, for instance, to dose two gases such as oxygen (gas source 1) and gas containing NO (gas source 2). The pressure or flow in the nose area (respiratory pressure or breathing gas flow) is recorded continuously via the pressure measuring line 6, on the basis of which the breathing curve is determined. The signal to initiate the triggering can be selected at will, for example, at the beginning of the inspiration (change from positive pressure to negative pressure, for instance, negative pressure of 0.1 mbar) or at a freely selectable pressure or flow during the inspiration. The actual dosing of the gases from gas sources 1 and 2 is done via the separate gas line 5, so that the pressure recording for purposes of determining the breathing curve is hardly or not at all disrupted. As a result, the breathing curve or the inspiration curve can also be recorded during the dosing of the gas.

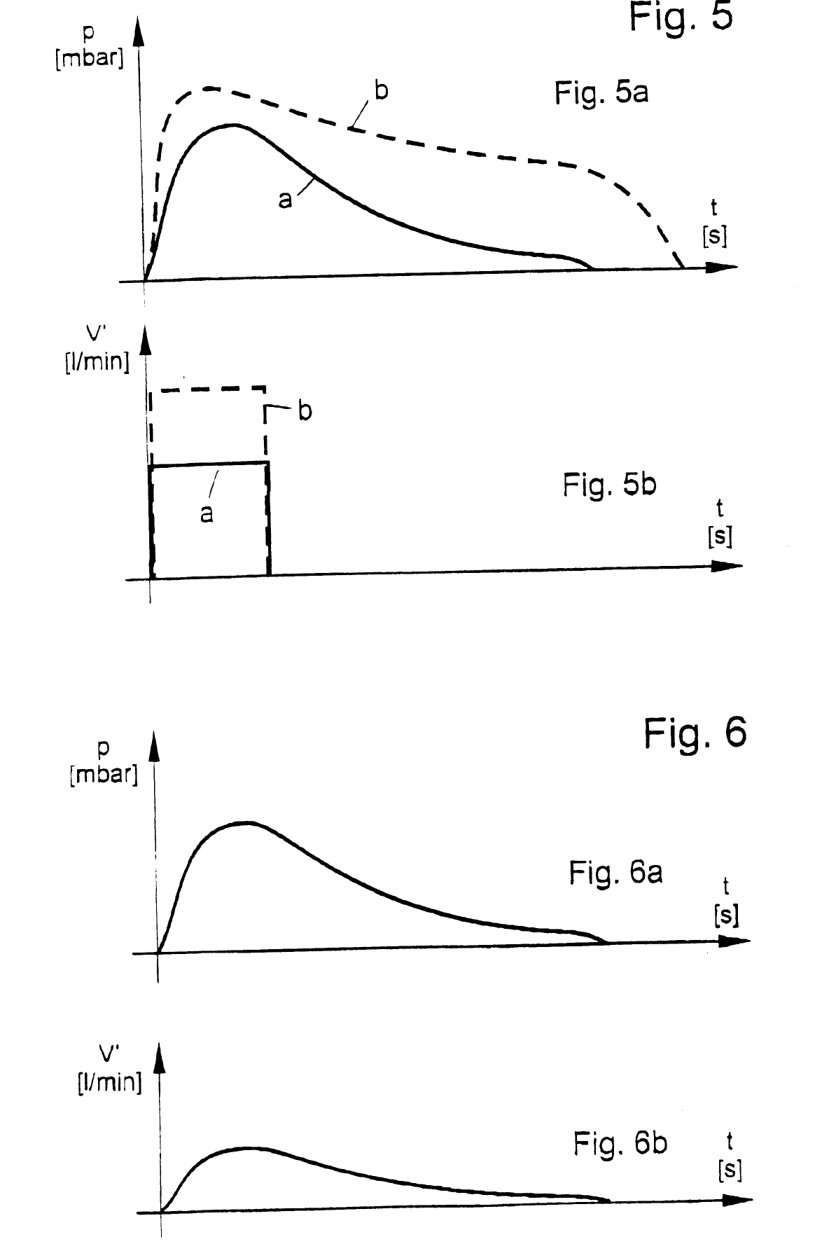

FIG. 5 shows an example of how the dosed amount of gas or the gas volume flow V′ is adapted (FIG. 5b) as a function of differing states (a, b) of the patient (FIG. 5a). An adjustable valve changes the gas flow of the dosed gas in such a way that an increased gas surge takes place at a constant gas volume flow V′ (gas spike) at the time of state b.

FIG. 6 shows an adaptation of a variable gas volume flow V′ of a dosed gas (FIG. 6b) to the ascertained breathing curve (FIG. 6a).

### REFERENCE NUMERALS

a rest

b exertion

c triggering threshold

1 gas source 1

2 gas source 2

3, 4 adjustable valve (for instance, solenoid valve)

5 gas line

6 pressure measuring line

7 patient

8 sensor (for example, pressure sensor)

9, 10, 11 control line

12 control unit

1. A gas-supply system for the inhalation treatment of humans or mammals, entailing controlled dosing of at least one gas, characterized by a tidal volume-dependent regulation of the gas dosing.

2. The gas-supply system according to claim 1, characterized in that the gas-supply system has an additional gas line (6) fitted with a sensor (8) for purposes of measuring the respiratory pressure or breathing gas flow.

3. The gas-supply system according to claim 1 or 2, characterized in that a pressure sensor or flow sensor (8) is present for purposes of measuring a tidal volume curve.

4. The gas-supply system according to one of claims 1 through 3, characterized in that the sensor (8) is part of a control means of the gas dosing.

5. The gas-supply system according to one of claims 1 through 4, characterized in that the gas-supply system comprises a control means (12) that is connected to the sensor (8) and adjustable valves (3, 4) for dosing the gas.

6. The gas-supply system according to one of claims 1 through 5, characterized in that the gas-supply system comprises a gas source (1, 2) for oxygen and a gas containing NO; a gas containing NO and hydrogen; oxygen and hydrogen; oxygen and helium; oxygen, a gas containing NO and hydrogen; oxygen, a gas containing NO and helium; oxygen, carbon dioxide and helium; or oxygen, a gas containing NO, carbon dioxide and hydrogen.

7. A method for operating gas-supply systems to supply gas to humans or mammals, characterized in that a sensor (8) is employed to measure a tidal volume curve and a controlled gas dosing takes place as a function of a measured tidal volume curve.

8. The method according to claim 7, characterized in that a gas-supply system according to one of claims 1 through 6 is employed.

9. The method according to claim 7 or 8, characterized in that a tidal volume curve is interpolated from measured pressure values and the interpolated tidal volume curve serves to regulate the dosing of at least one gas or aerosol.

10. The method according to one of claims 7 through 9, characterized in that the gas dosing is inspiration-synchronized and it takes place by means of program control, sensor control or combined program-sensor control, or else in sequences.

11. The use of a gas-supply system according to one of claims 1 through 6 for purposes of supplying gas to ventilated or spontaneously breathing patients.

12. The use according to claim 11 in order to supply gas to COPD patients.

13. The use according to claim 11 or 12, characterized in that oxygen and gas containing NO; oxygen, gas containing NO and helium; oxygen, gas containing NO, carbon dioxide and helium; oxygen, carbon dioxide and helium; or oxygen, gas containing NO and hydrogen are dosed.
